(19) United States
(12) Patent Application Publication
(10) Pub. No.: US 2021/0069144 A1
(43) Pub. Date: Mar. 11, 2021

(54) ISOTOPICALLY MODIFIED COMPOSITION AND THERAPEUTIC USES THEREOF

(71) Applicant: Retrotope, Inc., Los Altos, CA (US)

(72) Inventor: Mikhail S. SHCHEPINOV, Oxford (GB)

(21) Appl. No.: 17/045,094

(22) PCT Filed: Apr. 4, 2019
(86) PCT No.: PCT/US2019/025646

§ 371 Date: Oct. 2, 2020

Related U.S. Application Data
(60) Provisional application No. 62/652,855, filed on Apr. 4, 2018.

(51) Int. Cl. A61K 31/231; A61P 25/28; A61K 31/201

(52) U.S. Cl. CPC A61K 31/231; A61K 31/201; A61P 25/28

(57) ABSTRACT

Isotopically modified polyunsaturated fatty acid or ester thereof, such as 11,11-D2-linoleic acid or its ethyl ester, and uses of the same for treating or ameliorating the symptoms of Friedreich's ataxia arm disclosed.

### BACKGROUND

The present disclosure relates to the field of biochemistry and chemistry. Some embodiments relate to deuterated polyunsaturated acids or esters thereof and their therapeutic uses.

Friedreich's ataxia (FRDA) is an autosomal-recessive disorder that causes progressive ataxia, scoliosis, sensory loss, and hypertrophic cardiomyopathy. FRDA is caused by homozygous gene expansion in the frataxin gene (FXN), which decreases the expression of the mitochondrial, iron-binding protein frataxin, thus impairing the formation of cytosolic and mitochondrial iron-sulfur-cluster-containing enzymes. The ensuing iron imbalance through Fenton chemistry initiates lipid peroxidation (LPO), which leads to increased oxidative stress and mitochondrial dysfunction. LPO represents a particularly toxic type of oxidative damage to mitochondrial and other lipid membranes attributed to its unique, self-sustaining chain reaction format. Hill et al., “Isotope-reinforced polyunsaturated fatty acids protect yeast cells from oxidative stress,” Free Radic Biol Med 2011, 50:130-138.

There are no approved drugs to treat FRDA patients, and no therapies have been shown to alter the course of the disease. The search for a treatment for FRDA and other related mitochondrial disorders has focused on antioxidant-based therapies, agents to increase frataxin levels, chelators, and protein and gene replacement therapies. There exist a need for FRDA therapies having non-antioxidant mechanism of action.

Isotopically modified polyunsaturated fatty acids (PUFAs) and ester, thioester, amide, or other prodrug thereof, in particular bis-allyl-deuterated homologues, can make polyunsaturated fatty acids more resistant to the rate-limiting step of LPO. Moreover, relatively low levels of isotopically modified polyunsaturated fatty acid and ester, thioester, amide, or other prodrug thereof in lipid membranes efficiently quench the chain reaction, protecting polyunsaturated fatty acids through a non-antioxidant mechanism.

### SUMMARY

Some embodiments of the present disclosure relate to a method of treating a subject having Friedreich's ataxia, comprising: administering to the subject an effective amount of an isotopically modified polyunsaturated acid (PUFA) or an ester, thioester, amide, or other prodrug thereof, or combinations thereof. In some embodiments, the isotopically modified PUFA is D2-linoleic acid, for example, 11,11-D2-linoleic acid. In one particular embodiment, the deuterated compound administered to the subject is 11,11-D2-linoleic acid ethyl ester. The deuterated PUFA or ester, thioester, amide, or other prodrug thereof as disclosed herein inhibits lipid peroxidation and may be used to reduce cellular damage and recover mitochondrial function in degenerative diseases such as Friedreich's ataxia.

Some embodiments of the present disclosure relate to a method of treating a subject having Friedreich's ataxia, comprising selecting for treatment a subject having Friedreich's ataxia; and administering an amount of 11,11-D2-linoleic acid and/or an ester thereof to the subject in need thereof.

Some embodiments of the present disclosure relate to a method of treating a subject having Friedreich's ataxia, comprising selecting for treatment a subject having Friedreich's ataxia; administering an amount of 11,11-D2-linoleic acid or an ester thereof to the subject in need thereof; and advising the subject to take 11,11-D2-linoleic acid and/or the ester thereof with food or between meals.

In some embodiments of the methods described herein, the subject with Friedreich's ataxia may have mutations in the frataxin (FXN) gene. In some further embodiments, the mutations comprise expanded GAA repeat mutation in the FXN gene, for example, in intron 1 of FXN gene. In any of the embodiments described herein, the 11,11-D2-linoleic acid ester is an ethyl ester.

### BRIEF DESCRIPTION OF THE DRAWINGS

FIG. 1 shows the mean plasma concentrations of 11,11-D2-linoleic acid ethyl ester (D2-LA) for cohort 1 (1.8 g QD) and mean plasma concentrations of D2-LA for cohort 2 (4.5 g BID, 9 g/day).

FIG. 2 shows the mean concentrations of non-deuterated ethyl linoleate (H2-LA) in plasma: low dose (1.8 g QD) and high dose (4.5 g BID).

FIG. 3 shows the mean (SD) AUC-₀₋₂₄ₕ of D2-LA in plasma: low dose (1.8 g QD) and high dose (4.5 g BID).

FIG. 4 shows change in peak workload and VO₂max in subjects treated with comparator and D2-LA.

### DETAILED DESCRIPTION

Embodiments of the present disclosure relate to use of isotopically modified polyunsaturated acid (PUFA) or an ester, thioester, amide, or other prodrug thereof, or combinations thereof for treating or ameliorating Friedreich's ataxia (FRDA). FRDA is an autosomal recessive genetic disease. Patients with FRDA may demonstrate various symptoms, including difficulty walking, a loss of sensation in the arms and legs, impaired speech that worsens over time. Many patients have a form of heart disease called hypertrophic cardiomyopathy. The treatment with isotopically modified PUFA, in particular 11,11-D2-linoleic acid or an ester thereof. In one particular embodiment, the isotopically modified PUFA is 11,11-D2-linoleic acid ethyl ester. The study has shown that relatively low levels of 11,11-D2-linoleic acid ethyl ester in lipid membranes efficiently quench the chain reaction of LPO, protecting polyunsaturated fatty acids (PUFAs) in the mitochondrial and other lipid membrane from oxidative damages.

Some embodiments of the present disclosure relate to a method of treating a subject having Friedreich's ataxia, comprising selecting for treatment a subject having Friedreich's ataxia; and administering an effective amount of 11,11-D2-linoleic acid or an ester thereof to the subject in need thereof.

Some embodiments of the present disclosure relate to a method of treating a subject having Friedreich's ataxia, comprising administering an effective amount of 11,11-D2-linoleic acid or an ester thereof to the subject in need thereof; and advising the subject to take 11,11-D2-linoleic acid or the ester thereof with food or between meals.

In some embodiments of the method described herein, the method may include selecting for treatment a subject with Friedreich's ataxia. In some such embodiments, the subject with Friedreich's ataxia may have mutations in the frataxin (FXN) gene. In some further embodiments, the mutations comprise expanded GAA repeat mutation in the FXN gene, for example, in intron 1 of FXN gene. In some such embodiment, the subject has a FRDA onset at equal or less than 30, 25, or 20 years of age. The subject may be evaluated using the Friedreich Ataxia Rating Scale (FARS) prior to the treatment. The FARS comprises a measure of ataxia, an activities of daily living (ADL) subscale and a neurological subscale (FARS-Neuro). The scores can be added to make a total score ranging from 0 to 159. A higher score indicates a greater level of disability. Fahey et al., J Neurol Neurosurg Psychiatry 2007; 78:411-413. In some embodiments, the subject has a FARS-Neuro score of from 10 to about 120, from about 15 to about 110, from about 20 to about 100, from about 25 to about 95, or from about 30 to about 90. In some further embodiment, the subject may have a body mass index (BMI) from about 15 kg/m² to about 35 kg/m², or from about 16 kg/m² to about 33 kg/m², or from about 18 kg/m² to about 30 kg/m².

In some embodiments of the method described herein, the therapeutically effective amount of 11,11-D2-linoleic acid or the ester thereof administered to the subject is about 0.1 g, 0.2 g, 0.5 g, 1.0 g, 1.5 g, 2.0 g, 2.5 g, 3.0 g, 3.5 g, 4.0 g, 4.5 g, 5.0 g, 5.5 g, 6.0 g, 6.5 g, 7.0 g, 7.5 g, 8.0 g, 8.5 g, 9.0 g, 9.5 g, 10 g, 10.5 g, 11 g, 11.5 g, 12 g, 12.5 g, 13 g, 13.5 g, 14 g, 14.5 g, 15 g, 15.5 g, 16 g, 16.5 g, 17 g, 17.5 g 18 g, 18.5 g, 19 g, 19.5 g, or 20 g, or a range defined by any of the two preceding values. In some embodiments, the amount of 11,11-D2-linoleic acid or ester thereof administered to the subject is from about 0.1 g to about 20 g, from about 1 g to about 10 g, from 2 g to about 5 g. In some further embodiments, the amount of 11,11-D2-linoleic acid or ester administered is from about 1.8 g to about 4.5 g. In some embodiments, 11,11-D2-linoleic acid or ester is in a single unit dosage form. In some other embodiments, 11,11-D2-linoleic acid or ester is in two or more unit dosage forms (i.e., a divided dose). For example, where a dose is about 5 g, it may be provided in the form of four or five tablets, each containing about 1.25 g or 1 g of 11,11-D2-linoleic acid or ester thereof. In some such embodiments, a dose of 1 g to 10 g comprises administering 1, 2, 3, 4 or 5 unit dosage forms each comprising from about 1 g to about 2 g of 11,11-D2-linoleic acid or ester thereof, or about 2, 3, or 4 unit dosage forms each comprising from about 0.5 g to about 2.5 g of 11,11-D2-linoleic acid or ester thereof. In another example, a dose of 2 g to 5 g comprises administering 1, 2, 3, 4 or 5 unit dosage forms each comprising from about 1 g to about 2 g of 11,11-D2-linoleic acid or ester thereof. In some embodiments, the unit dosage form is a tablet, a capsule, a pill, or pellets. In some further embodiment, the unit dosage form for oral administration, i.e., oral dosage form.

In some embodiments of the method described herein, 11,11-D2-linoleic acid or the ester thereof may be administered once per day. In some other embodiments, 11,11-D2-linoleic acid or the ester thereof may be administered two or more times per day, for example, twice a day or three times a day. In some embodiments, the therapeutically effective amount of 11,11-D2-linoleic acid or the ester thereof administered per day is about 1.0 g, 2.0 g, 3.0 g, 3.5 g, 4.0 g, 4.5 g, 5.0 g, 5.5 g, 6.0 g, 6.5 g, 7.0 g, 7.5 g, 8.0 g, 8.5 g, 9.0 g, 9.5 g, 10 g, 10.5 g, 11 g, 11.5 g, 12 g, 12.5 g, 13 g, 13.5 g, 14 g, 14.5 g, 15 g, 15.5 g, 16 g, 16.5 g, 17 g, 17.5 g 18 g, 18.5 g, 19 g, 19.5 g, 20 g, 25 g, 30 g, 35 g, 40 g, 45 g, or 50 g, or a range defined by any of the two preceding values. In some such embodiments, the amount of 11,11-D2-linoleic acid or the ester thereof administered per day is from about 1 g to about 50 g, from about 2 g to about 40 g, from about 3 g to about 30 g, from about 4 g to about 20 g, or from about 5 g to about 0 g. In one embodiment, the amount of 11,11-D2-linoleic acid or the ester thereof administered per day is from about 2 g to about 10 g. In another embodiment, the amount of 11,11-D2-linoleic acid or the ester thereof administered per day is from about 1.8 g to about 9 g.

In some embodiments of the method described herein, 11,11-D2-linoleic acid or the ester thereof may be administered for at least 1 week, 2 weeks, 3 weeks, 4 weeks, 5 weeks, 6 weeks, 7 weeks or 8 weeks. In some embodiments, the method further comprises detecting the steady state plasma level of 11,11-D2-linoleic acid or the ester thereof. In some such embodiments, the plasma level of 11,11-D2-linoleic acid or the ester thereof reaches a steady state after 1, 2, 3 or 4 weeks.

In some embodiments of the method described herein, the method ameliorates the symptoms of FRDA and improves certain functional parameters in FRDA subject, including but not limited to improved Friedreich's Ataxia Rating Scale (FARS)-Neurological score (FARS-Neuro), the timed 25-foot walk (T25FW), and cardiopulmonary exercise testing (CPET) endpoints such as peak oxygen consumption (VO₂ max) and peak workload. In some such embodiments, the improvement of the functional parameters in FRDA subject is at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200%. For example, the reduction of the FARS-Neuro score from 80 to 60 is a 25% improvement. The T25FW from 5 min to 4 min is a 20% improvement. In some further embodiments, the method improves the peak workload from baseline by at least about 0.01 watts/kg, 0.02 watts/kg, 0.03 watts/kg, 0.04 watts/kg, 0.05 watts/kg, 0.06 watts/kg, 0.07 watts/kg, 0.08 watts/kg, 0.09 watts/kg, 0.10 watts/kg, 0.11 watts/kg, 0.12 watts/kg, 0.13 watts/kg, 0.14 watts/kg, 0.15 watts/kg, 0.16 watts/kg, 0.17 watts/kg, 0.18 watts/kg, 0.19 watts/kg, 0.20 watts/kg, 0.21 watts/kg, 0.22 watts/kg, 0.23 watts/kg, 0.24 watts/kg, or 0.25 watts/kg. In one embodiment, the method improves the peak work load by about 25%. In some further embodiments, the method improves the peak oxygen consumption (VO₂max) from baseline by at least about 0.01 L/min, 0.02 L/min, 0.03 L/min, 0.04 L/min, 0.05 L/min, 0.06 L/min, 0.07 L/min, 0.08 L/min, 0.09 L/min, 0.1 L/min, 0.11 L/min, 0.12 L/min, 0.13 L/min, 0.14 L/min, 0.15 L/min, 0.16 L/min, 0.17 L/min, 0.18 L/min, 0.19 L/min, 0.2 L/min, 0.21 L/min, 0.22 L/min, 0.23 L/min, 0.24 L/min, or 0.25 L/min. In one embodiment, the method improves the VO₂max by about 17.5%. In some further embodiments, the method decreases the FARS-Neuro score by at least 1, 2, 3, 4, 5, 6, 7, 8, 9, 10, 11, 12, 13, 14, 15, 16, 17, 18, 19, 20, 21, 22, 23, 24, or 25 points. In one embodiment, the method decreases the FARS-neuro score by about 5 points. In another embodiment, the method decreases the FARS-neuro score by about 12 points.

In some embodiments of the method described herein, the subject may also ingest a non-isotopically modified polyunsaturated fatty acid, or ester, thioester, amide, or other prodrug thereof, either concurrently, prior to, or subsequent to the administration of the 11,11-D2-linoleic acid or the ester thereof. In some such embodiments, the subject may also ingest a non-isotopically modified polyunsaturated fatty acid, or a non-isotopically modified polyunsaturated fatty acid ester, or a combination thereof. In some embodiments, the amount of 11,11-D2-linoleic acid or the ester thereof is at least about 5%, 10%, 15%, 20%, 25%, 30%, 35%, 40%, 45%, 50% or greater of the total amount of the polyunsaturated fatty acids and polyunsaturated fatty acid esters delivered the subject. In some other embodiments, the amount of 11,11-D2-linoleic acid or the ester thereof is equal to less than about 5%, 4%, 3%, 2%, 1%, or 0.5% of the total amount of the polyunsaturated fatty acids and polyunsaturated fatty acid esters delivered to the subject. In some such embodiments, the non-isotopically modified PU FA or derivatives thereof may be taken concurrently, prior to, or subsequent to the administration of isotopically modified PUFA (e.g., 11,11-D2-linoleic acid or the ester thereof). In some embodiments, non-isotopically modified and isotopically modified PUFAs may be in a single dosage form. In some embodiments, the single dosage form is selected from the group consisting of a pill, a tablet, and a capsule.

In some embodiments of the method described herein, 11,11-D2-linoleic acid or the ester thereof may be administered with at least one antioxidant. In some such embodiments, the antioxidant is selected from the group consisting of Coenzyme Q, idebenone, mitoquinone, mitoquinol, vitamin E, and vitamin C, and combinations thereof. In some such embodiments, the at least one antioxidant may be taken concurrently, prior to, or subsequent to the administration of 11,11-D2-linoleic acid or the ester thereof. In some embodiments, the antioxidant and 11,11-D2-linoleic acid or the ester thereof may be in a single dosage form. In some embodiments, the single dosage form is selected from the group consisting of a pill, a tablet, and a capsule.

In any of the embodiments, the 11,11-D2-linoleic acid ester may be an alkyl ester, for example, ethyl ester. In some other embodiments, the ester is a glyceride, for example a triglyceride, a diglyceride, or a monoglyceride. In any of the embodiments, the administration of a deuterated PUFA or ester thereof may also include the administration of both the PUFA and the ester, for example, both 11,11-D2-linoleic acid and its ethyl ester.

### Definition

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, formulation, or device, the term “comprising” means that the compound, composition, formulation, or device includes at least the recited features or components, but may also include additional features or components.

As used herein, common abbreviations are defined as follows:
AE Adverse event
ANOVA analysis of variance
AUC area under the concentration-time curve
BID Twice daily
BMI Body mass index
Cₘₐₓ Maximum plasma concentration
CPET Cardiopulmonary exercise testing
FRDA Friedreich's ataxia
FARS Friedriech's Ataxia Rating Scale
FARS-Neuro Friedriech's Ataxia Rating Scale—Neurological
LPO Lipid Peroxidation
RBC Red blood cell
PK Pharmacokinetics
PUFA Polyunsaturated Fatty Acid
QD Once daily
SD Standard deviation
t_{1/2} Apparent plasma terminal elimination half life
Tₗₐₛₜ Time to last measurable concentration
T25FW Timed 25-foot walk

The term “about” as used herein, refers to a quantity, value, number, percentage, amount, or weight that varies from the reference quantity, value, number, percentage, amount, or weight by a variance considered acceptable by one of ordinary skill in the art for that type of quantity, value, number, percentage, amount, or weight. In various embodiments, the term “about” refers to a variance of 20%, 15%, 10%, 9%, 8%, 7%, 6%, 5%, 4%, 3%, 2% or 1% relative to the reference quantity, value, number, percentage, amount, or weight.

The term “oral dosage form,” as used herein, has its ordinary meaning as understood by those skilled in the art and thus includes, by way of non-limiting example, a formulation of a drug or drugs in a form administrable to a human, including pills, tablets, cores, capsules, caplets, loose powder, solutions, and suspensions.

The terms “pharmacokinetic profile” or “pharmacokinetics,” as used herein, have their ordinary meaning as understood by those skilled in the art and thus include, by way of non-limiting example, a characteristic of the curve that results from plotting concentration (e.g. blood plasma, serum or tissue) of a drug over time, following administration of the drug to a subject. A pharmacokinetic profile thus includes a pharmacokinetic parameter or set of parameters that can be used to characterize the pharmacokinetics of a particular drug or dosage form when administered to a suitable population. In some embodiments, the suitable population may be defined as patients with renal impairment, patients with hepatic impairment, geriatrics, or pediatrics, etc. Various pharmacokinetic parameters are known to those skilled in the art, including area under the concentration vs. time curve (AUC), area under the concentration time curve from time zero until last quantifiable sample time (AUC₀₋ₜ), area under the concentration time curve from time zero extrapolated to infinity (AUC_{0-∞}), area under the concentration time curve over the steady state dosing interval (AUC_{SS}) or from time zero to twelve hours (AUC₀₋₁₂) for twice-daily dosing, maximum concentration (e.g. blood plasma/serum) after administration (Cₘₐₓ), minimum concentration (e.g. blood plasma/serum) after administration (Cₘᵢₙ), and time to reach maximum concentration (e.g. blood plasma/serum) after administration (Tₘₐₓ). AUCₗₐₛₜ indicates the area under the blood plasma concentration vs. time curve from the time of administration until the time of the last quantifiable concentration. Pharmacokinetic parameters may be measured in various ways known to those skilled in the art, e.g., for single dose or steady-state. Differences in one or more of the pharmacokinetic parameters (e.g., Cₘₐₓ) may indicate pharmacokinetic distinctness between two formulations or between two methods of administration.

The terms “patient” or “subject” refers to a human patient.

As used herein, the act of “providing” includes supplying, acquiring, or administering (including self-administering) a composition described herein.

As used herein, the term “administering” a drug includes an individual obtaining and taking a drug on their own. For example, in some embodiments, an individual obtains a drug from a pharmacy and self-administers the drug in accordance with the methods provided herein.

The term “therapeutically effective amount” as used herein, refers to an amount of an isotopically modified compound described herein sufficient to treat, ameliorate Friedreich's ataxia (FRDA), or to exhibit a detectable therapeutic effect. The effect may be detected by any means known in the art. In some embodiments, the precise effective amount for a subject can depend upon the subject's body weight, size, and health; the nature and extent of the condition; and the therapeutic or combination of therapeutics selected for administration. Therapeutically effective amounts for a given situation may be determined by routine experimentation that is within the skill and judgment of the clinician. In some embodiments, isotopically modified compound is a polyunsaturated acid (PUFA) or an ester, thioester, amide, or other prodrug thereof, or combinations thereof for treating, or ameliorating Friedreich's ataxia (FRDA). In some further embodiment, the isotopically modified PUFA is 11,11-D2-linoleic acid or an ester thereof.

As used herein, the term “with food” is defined to mean, in general, the condition of having consumed food during the period between from about 1 hour prior to the administration of the isotopically modified compound described herein to about 2 hours after the administration of such compound. In some embodiments, the food is a solid food with sufficient bulk and fat content that it is not rapidly dissolved and absorbed in the stomach. Preferably, the food is a meal, such as breakfast, lunch, or dinner. In some embodiments, the food is at least about 100 calories, about 200 calories, about 250 calories, about 300 calories, about 400 calories, about 500 calories, about 600 calories, about 700 calories, about 800 calories, about 900 calories, about 1000 calories, about 1250 calories, about 1500 calories.

The pharmaceutical composition described herein are preferably provided in unit dosage form. As used herein, a “unit dosage form” is a composition/formulation containing an amount of a compound that is suitable for administration to an animal, preferably mammal subject, in a single administration, according to good medical practice. The preparation of a single or unit dosage form however, does not imply that the dosage form is administered once per day or once per course of therapy, or that the unit dosage form contains all of the dose to be administered at a single time. Such dosage forms are contemplated to be administered once, twice, thrice or more per day, and may be given more than once during a course of therapy, though a single administration is not specifically excluded. In addition, multiple unit dosage forms may be administered at substantially the same time to achieve the full dose intended (e.g., two or more tablets may be swallowed by the patient to achieve a complete dose). The skilled artisan will recognize that the formulation does not specifically contemplate the entire course of therapy and such decisions are left for those skilled in the art of treatment rather than formulation.

In any of the embodiments described herein, methods of treatment can alternatively entail use claims, such as Swiss-type use claims. For example, a method of treating a subject having FRDA can alternatively entail the use of a compound in the manufacture of a medicament for the treatment of FRDA, or a compound for use in the treatment of FRDA.

Those skilled in the art will understand that pharmacokinetic parameters may be determined by comparison to a reference standard using clinical trial methods known and accepted by those skilled in the art, e.g., as described in the examples set forth herein. Since the pharmacokinetics of a drug can vary from patient to patient, such clinical trials generally involve multiple patients and appropriate statistical analyses of the resulting data (e.g., ANOVA at 90% confidence). Comparisons of pharmacokinetic parameters can be on a dose-adjusted basis, as understood by those skilled in the art.

### Pharmacokinetics and Metabolite Analysis

In some embodiments of the method described herein, the method further comprises measuring the concentration of 11,11-D2-linoleic acid or ester thereof (D2-LA) in the plasma and/or red blood cells (RBCs). Such measurement may be used to determine the minimum administration period for the dose of 11,11-D2-linoleic acid or ester to reach a steady state, dose optimization, and/or the therapeutic window. In some embodiments, the plasma D2-LA concentration reaches a steady state after about 4 weeks. In some further embodiments, the steady state plasma 24-hour AUC of 11,11-D2-linoleic acid or the ester thereof is from about 100 μg*h/mL to about 10,000 μg*h/mL, from about 500 μg*h/mL to about 9000 μg*h/mL, from about 1000 μg*h/mL to about 8000 μg*h/mL, from about 1250 μg*h/mL to about 7500 μg*h/mL, or from about 1500 μg*h/mL to about 7000 μg*h/mL. In some further embodiments, the steady state plasma 24-hour AUC of 11,11-D2-linoleic acid or the ester thereof is from about 1,527±445 μg*h/mL to about 6800±918 μg*h/mL. In some embodiments, the steady state RBC concentration of 11,11-D2-linoleic acid or the ester thereof is from about 1 μg/mL to about 100 μg/mL, from about 2 μg/mL to about 80 μg/mL, from about 5 μg/mL to about 75 μg/mL, from about 7.5 μg/mL to about 50 μg/mL, from about 10 μg/mL to about 40 μg/mL, from about 15 μg/mL to about 30 μg/mL, or from about 20 μg/mL to about 25 μg/mL. In one embodiment, the steady state plasma concentration of 11,11-D2-linoleic acid or the ester thereof is from about 14.6±8.7 μg/mL to about 26.1±19.2 μg/mL. In some embodiments, the concentration of 11,11-D2-linoleic acid or the ester thereof in the red blood cells is about 1%, 2%, 5%, 7.5%, 10%, 12.5%, 15%, 17.5%, 20%, 22.5%, 25%, 27.5%, 30%, 35%, 40%, 45%, 50%, 55%, 60%, 65%, 70%, or 75% of total linoleic acid or the ester thereof, or a range defined by any of the two preceding values. In some embodiments, the concentration of 11,11-D2-linoleic acid or the ester thereof in the red blood cells is about 5% to about 30% of total linoleic acid or the ester thereof. As described herein, the total linoleic acid or ester thereof refers to the total of both deuterated and non-deuterated linoleic acid or ester thereof. As described herein, the plasma or RBC concentration of 11,11-D2-linoleic acid or the ester thereof refers to the combined concentration of both free acid form and ester form of 11,11-D2-linoleic acid.

In some embodiments, 11,11-D2-linoleic acid or the ester thereof has a plasma Tₘₐₓ of about 8 hours to about 10 hours.

A primary metabolite of 11,11-D2-linoleic acid or ester thereof is an enzymatic elongation/desaturation brain-penetrant, 13,13-D2-arachidonic acid (D2-AA). In some embodiments of the method described herein, the method further comprises detecting the metabolite of 11,11-D2-linoleic acid or the ester thereof in the plasma, for example, measuring the concentration of 13,13-D2-arachidonic acid in the plasma and/or red blood cells (RBCs). In some such embodiments, the steady state plasma concentration of 13,13-D2-arachidonic acid is from about 1 μg/mL to about 50 μg/mL, from about 2 μg/mL to about 40 μg/mL, from about 3 μg/mL to about 30 μg/mL, or from about 4 μg/mL to about 25 μg/mL. In one embodiment, the steady state plasma concentration of 13,13-D2-arachidonic acid is from about 3.86 μg/mL to about 22 μg/mL.

Adverse events (AE)

Ingestion of high dose of PUFA or isotopically modified PUFA may causes undesirable gastrointestinal adverse events, for example, steatorrhea. In some instances, subjects with low BMI are more subject to gastrointestinal AEs, for example, subjects with BMI equal or less than 18 kg/m², 17 kg/m², 16 kg/m² or 15 kg/m². In these subjects, it is advisable to administer no more than 10 g, 9 g, 8 g, 7 g, 6 g, 5 g, 4 g, 3 g, 2 g, or 1 g of PUFA per dose (including both isotopically modified and non-isotopically modified PUFAs). In some further embodiments, it is advisable to administer no more than 5 g, 4 g, 3 g, 2 g, 1 g, or 0.5 g of PUFA per dose to a subject with a BMI less than 17 or 16 kg/m². Some embodiments of the present disclosure relate to a method of treating a subject having Friedreich's ataxia, comprising administering 11,11-D2-linoleic acid or an ester thereof to the subject in need thereof, and advising the subject to take 11,11-D2-linoleic acid or the ester thereof with food or between meals. For example, in some embodiments, the food can be consumed at any time during the period between from about 1 hours prior to the administration of 11,11-D2-linoleic acid or the ester thereof to about 2 hours after the administration of such compound. In some embodiments, the administration of 11,11-D2-linoleic acid or the ester to the patient is immediately after the consumption of food or a meal (e.g., within about 1 minute after food consumption) up to about 2 or 3 hour after food consumption. In some embodiments, 11,11-D2-linoleic acid or the ester thereof is administered between breakfast and lunch, or between lunch and dinner.

### Kits

Some additional embodiments of the present disclosure relate to kits comprising a pharmaceutical composition, prescribing information, and a container, wherein the pharmaceutical composition comprises a therapeutically effective amount of an isotopically modified compound described herein. In some embodiments, isotopically modified compound is a polyunsaturated acid (PUFA) or an ester, thioester, amide, or other prodrug thereof, or combinations thereof for treating, or ameliorating Friedreich's ataxia (FRDA). In some further embodiment, the isotopically modified PUFA is 11,11-D2-linoleic acid and/or an ester thereof. In one particular embodiment, the isotopically modified PUFA is 11,11-D2-linoleic acid ethyl ester. In some embodiments, the prescribing information advises a subject to take the pharmaceutical composition with food, or take the pharmaceutical composition between meals. The kit may include one or more unit dosage forms comprising 11,11-D2-linoleic acid or the ester thereof. The unit dosage forms may be of an oral formulation. For example, the unit dosage forms may comprise pills, tablets, or capsules. The kit may include a plurality of unit dosage forms. In some embodiments, the unit dosage forms are in a container. In some embodiments, the dosage forms are single oral dosage forms comprising 11,11-D2-linoleic acid or the ester thereof, e.g., the ethyl ester.

The methods, compositions and kits disclosed herein may include information. The information may be in a form prescribed by a governmental agency regulating the manufacture, use, or sale of pharmaceuticals, which notice is reflective of approval by the agency of the form of the drug for human or veterinary administration. Such information, for example, may be the labeling approved by the U.S. Food and Drug Administration for prescription drugs, or the approved product insert. The information can include required information regarding dose and dosage forms, administration schedules and routes of administration, adverse events, contraindications, warning and precautions, drug interactions, and use in specific populations (see, e.g., 21 C.F.R. § 201.57 which is incorporated herein by reference in its entirety), and in some embodiments is required to be present on or associated with the drug for sale of the drug. In some embodiments, a kit is for sale of a prescription drug requiring the approval of and subject to the regulations of a governmental agency, such as the Food and Drug Administration of the United States. In some embodiments, the kit comprises the label or product insert required by the agency, such as the FDA, for sale of the kit to consumers, for example in the U.S. In preferred embodiments, the information instructs an individual to take 11,11-D2-linoleic acid or the ester thereof between meals, or with food, in order to reduce possible adverse event(s), for example gastrointestinal adverse event(s).

Instructions and/or information may be present in a variety of forms, including printed information on a suitable medium or substrate (e.g., a piece or pieces of paper on which the information is printed), computer readable medium (e.g., diskette, CD, etc. on which the information has been recorded), or a website address that may be accessed via the internet. Printed information may, for example, be provided on a label associated with a drug product, on the container for a drug product, packaged with a drug product, or separately given to the patient apart from a drug product, or provided in manner that the patient can independently obtain the information (e.g., a website). Printed information may also be provided to a medical caregiver involved in treatment of the patient. In some embodiments, the information is provided to a person orally.

Some embodiments comprise a therapeutic package suitable for commercial sale. Some embodiments comprise a container. The container can be in any conventional shape or form as known in the art which is made of a pharmaceutically acceptable material, for example a paper or cardboard box, a glass or plastic bottle or jar, a re-sealable bag (e.g., to hold a “refill” of tablets for placement into a different container), or a blister pack with individual dosages for pressing out of the pack according to a therapeutic schedule. The container employed can depend on the exact dosage form involved, e.g., a conventional cardboard box would not generally be used to hold a liquid suspension. It is feasible that more than one container can be used together in a single package to market a single dosage form. For example, tablets may be contained in a bottle which is in turn contained within a box.

The information can be associated with the container, for example, by being: written on a label (e.g., the prescription label or a separate label) adhesively affixed to a bottle containing a dosage form described herein; included inside a container as a written package insert, such as inside a box which contains unit dose packets; applied directly to the container such as being printed on the wall of a box; or attached as by being tied or taped, e.g., as an instructional card affixed to the neck of a bottle via a string, cord or other line, lanyard or tether type device. The information may be printed directly on a unit dose pack or blister pack or blister card.

### Pharmaceutical Compositions

Some embodiments include pharmaceutical compositions comprising: (a) a safe and therapeutically effective amount of an isotopically modified compound described herein; and (b) a pharmaceutically acceptable carrier, diluent, excipient or combination thereof. In some embodiments, isotopically modified compound is a polyunsaturated acid (PUFA) or an ester, thioester, amide, or other prodrug thereof, or combinations thereof. In some further embodiment, the isotopically modified PUFA is 11,11-D2-linoleic acid or an ester thereof. In one particular embodiment, the isotopically modified PUFA is 11,11-D2-linoleic acid ethyl ester.

The compounds useful as described above can be formulated into pharmaceutical compositions for use in treatment of various conditions or disorders. Standard pharmaceutical formulation techniques are used, such as those disclosed in Remington's The Science and Practice of Pharmacy, 21st Ed., Lippincott Williams & Wilkins (2005), incorporated by reference in its entirety.

In addition to the selected compound useful as described above, some embodiments include compositions containing a pharmaceutically-acceptable carrier. The term “pharmaceutically-acceptable carrier”, as used herein, means one or more compatible solid or liquid filler diluents or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Pharmaceutically-acceptable carriers include, for example, solid or liquid fillers, diluents, hydrotropies, surface-active agents, and encapsulating substances. Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modem Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Per-oral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives or supplements. For example, the pharmaceutical composition is administered concomitantly with one or more antioxidants. In some embodiments, the antioxidant is selected from the group consisting of Coenzyme Q, idebenone, mitoquinone, mitoquinol, vitamin E, and vitamin C, and combinations thereof. In some such embodiments, at least one antioxidant may be taken concurrently, prior to, or subsequent to the administration of 11,11-D2-linoleic acid or the ester thereof. In some embodiments, the antioxidant and 11,11-D2-linoleic acid or the ester thereof may be in a single dosage form. In some embodiments, the single dosage form is selected from the group consisting of a pill, a tablet, and a capsule.

It will be understood by those of skill in the art that numerous and various modifications can be made without departing from the spirit of the present invention. Therefore, it should be clearly understood that the embodiments of the present invention disclosed herein are illustrative only and are not intended to limit the scope of the present invention. Any reference referred to herein is incorporated by reference for the material discussed herein, and in its entirety.

### EXAMPLE

A phase I/II double blind, comparator-controlled trial with 2 doses of 11,11-D2-linoleic acid ethyl ester in Friedreich's ataxia patients (9 subjects in each cohort) was conducted. Subjects were randomized 2:1 to receive either 11,11-D2-linoleic acid ethyl ester (1.8 or 9.0 g/day), or a matching dose of non-deuterated ethyl linoleate as comparator for 28 days.

The primary study objectives were to evaluate the safety and tolerability of orally administered 11,11-D2-linoleic acid ethyl ester. Additional study objectives included identifying the PK of 11,11-D2-linoleic acid ethyl ester following single and multiple oral dose administration and establishing an optimum dose of 11,11-D2-linoleic acid ethyl ester. Additional clinical testing consisted of the Friedreich's Ataxia Rating Scale (FARS)-Neurological point (FARS-Neuro), the timed 25-foot walk (T25FW) with an electronic sensor to measure multiaxis components of motion during the study, and cardiopulmonary exercise testing (CPET). The CPET was performed on an electronically braked cycle ergometer (Lode Corival, Groningen, The Netherlands). The protocol included: 5 minutes of rest; 3 minutes of an unloaded cycling warm-up; a progressive protocol with increased workload each minute (5-25 watts based on estimated functional capacity); 2 minutes of an unloaded cycling recovery; and 10 minutes of passive recovery. Heart rate (HR), blood pressure (BP), ratings of perceived exertion (RPE), pulse oximetry, and expired gases were monitored in accord with standard exercise testing guidelines. See Pescatello et al., Thompson P. ACSM's Guidelines for Exercise Testing and Prescription. Baltimore, Md.: Wolters Kluwer, 2014:114-141. Heart rate was measured by electrocardiography (Pulse Biomedical, King of Prussia, Pa.), BP was determined by auscultation, RPE was estimated using the standard 6 to 20 scale, and expired gases were collected through a silicone rubber mask (Hans Rudolph, Shawnee, Kans.) and analyzed continuously using a metabolic cart (Parvomedics, AQ5 Sandy, Utah) Vacumetrics (Ventura, Calif.). Endpoints of interest were peak oxygen consumption (VO₂ peak) and peak workload. All other variables were primarily assessed to monitor patient safety and progress during the exercise trial.

### Materials and Methods

The study included male or female participants, aged 18 to 50 years, with a FRDA onset at equal or less than 25 years of age, homozygosity for GAA repeat expansions in FXN, and FARS-Neuro range of 20 to 90 points. Participants were ambulatory (with or without assistive device), capable of performing other assessments/evaluations, with a body mass index (BMI) equal or less than 34 kg/m². Subjects were agreeable to dietary restriction coaching from a diet counselor to lower PUFA dietary intake, for maximal incorporation of 11,11-D2-linoleic acid ethyl ester. Sample size was selected to ensure 12 fully evaluable patients for active treatment and 6 patients for the comparator treatment. It was not powered, but considered adequate for a first-in-human trial of 11,11-D2-linoleic acid ethyl ester.

Study participants in cohort 1 were randomly assigned to receive either 11,11,D2-linoleic acid ethyl ester 1.8 g or inactive comparator QD for 28 days (2:1 drug vs. comparator), whereas subjects assigned to cohort 2 received either 11,11,D2-linoleic acid ethyl ester 4.5 g BID (9.0 g per day) or inactive comparator for 28 days (2:1, drug vs. comparator). A randomization schedule was generated before the first dosing period by Agility Clinical, Inc. (Carlsbad, Calif.) that followed their established standard operating procedures regarding generation, security, and distribution of the randomization scheme. Patients who provided informed consent and met all enrollment criteria were randomized into the study. In order to randomize a patient, the site contacted the unblinded statistician at Agility, Inc to identify which kit should be provided to the patient.

Patients were randomly assigned to receive one of two treatments (11,11,D2-linoleic acid ethyl ester or comparator) following a 2:1 ratio, respectively. The study sponsor, investigators, research pharmacy, and patients were blinded to the subject's treatment. Safety assessments included physical and neurological examinations, vital signs, triplicate 12-lead electrocardiograms, clinical laboratory tests (hematology, clinical chemistry, lipid profile, coagulation, and urinalysis), and adverse events (AEs). AEs were evaluated for incidence, severity, and relationship to study drug.

PK samples were obtained predose and at 0.5, 1, 1.5, 2, 4, 6, 8, 12, 16, and 24 hours following the first dose and 14, 28, 29, 30, and 31 days following enrollment. The samples taken on Days 29-31 represent Isotopically modified polyunsaturated fatty acid, ester, thioester, amide, or other prodrug thereof (e.g., 11,11,D2-linoleic acid ethyl ester) washout levels. D2-LA, linoleic acid (LA), deuterated arachidonic acid (D2-AA), and arachidonic acid (ARA) concentrations were measured in the samples.

### Analytical and PK Methods

Plasma concentrations of 11,11-D2-linoleic acid ethyl ester (combined free acid form and ester form) and non-deuterated analogs were determined by liquid chromatography and mass spectrometry (LCMS). Blood was collected in K2EDTA anticoagulant tubes, centrifuged to obtain plasma and red blood cells (RBCs), and frozen until analyzed. The samples were hydrolyzed to free fatty acids and analyzed by high performance LC-MS by Good Laboratory Practice validated methods. Low-intensity D2 and high-intensity H2 signals were measured with high accuracy at Ricerca Biosciences, using an AB SCIEX 6500 tandem-quadrupole MS. The ¹³C₂ isotopomer signal was used as a calibration standard for 11,11,D2-linoleic acid ethyl ester, a polydeuterated internal standard was utilized. The enzymatic elongation/desaturation metabolite, 13,13-D2-arachidonic acid (D2-AA), was also measured, in plasma and red blood cells (RBCs).

### Subject Demographics and Baseline Characteristics

Nineteen subjects were enrolled in the study, and 18 subjects completed all safety and efficacy measurements. One subject discontinued the study. The first patient was screened in September 2015, and the last subject completed follow-up in July 2016. Overall, 13 subjects received Isotopically modified polyunsaturated fatty acid, ester, thioester, amide, or other prodrug thereof (e.g., 11,11,D2-linoleic acid ethyl ester), and 6 subjects received inactive comparator. Age, sex, weight, and baseline FARST1 Neuro points are displayed in Table 1.

TABLE 1
Demographics of the clinical study

| | | Age (year) | | Baseline Weight | | Baseline FARS- |
|---|---|---|---|---|---|---|
| | n | Min-Max | | (kg) | | Neuro |
| | (subject) | (Median) | Sex | Min-Max (Median) | BMI | Min-Max (Median) |
| D2-LA | 13 | 18 to 48 | 8F:5M | 38.3 to 101.4 | 15.4 to 29.5 | 33 to 86 |
| | | (34) | | (60.0) | (22.93) | (66) |
| H2-LA | 6 | 23 to 47 | 2F:4M | 51.8 to 106.5 | 16.9 to 32.8 | 38 to 66 |
| | | (37) | | (77.0) | (25.30) | (48) |

### Safety Assessments

Overall, 11,11,D2-linoleic acid ethyl ester was well tolerated. The sole treatment emergent adverse event (TEAE) in more than 1 subject per treatment group in cohort 2 was diarrhea. Diarrhea was assessed by the investigators as “probably related to study drug” in 1 subject and “possibly related to study drug” in 3 subjects all in the drug group in cohort 2, as well as 1 subject in the comparator treatment group of cohort 2. One subject with a very low BMI of 15.4 in the high-dose group (cohort 2) discontinued the study within the first week because of steatorrhea. The patient was hospitalized as a precaution, but the episode was self-limited and resolved upon discontinuation of study drug. All other TEAEs were of mild-to-moderate severity, and the relationship to study drug was determined to be unlikely.

### PK Assessments

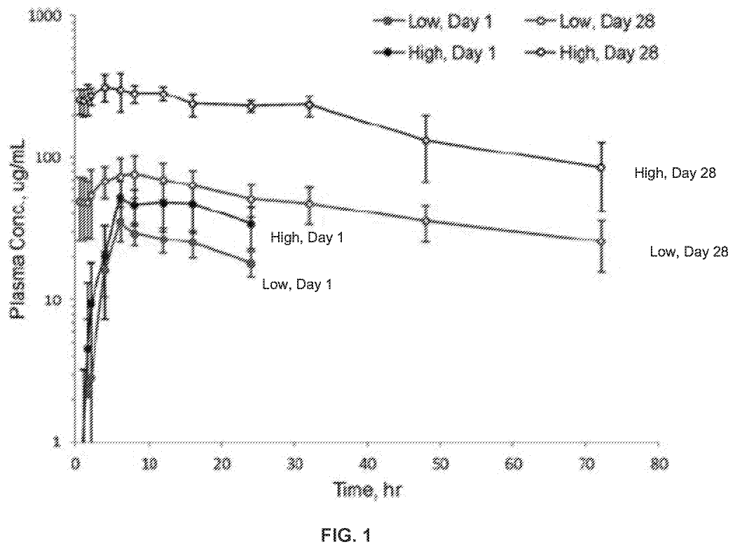

On days 1 and 28 only, cohort 2 subjects (high dose) were dosed at 4.5 g QD (vs. BID) for high resolution PK analysis. Plasma D2-LA levels were below quantitation for the first few hours after dosing in most subjects, rose to maximum concentrations at around 8 to 10 hours, and then fell slowly for the rest of the 24-hour dosing interval. Plasma D2-LA concentrations rose over the course of the 28-day study in both cohorts and appeared to be approaching a steady state by day 28 of dosing (FIG. 1). Mean plasma D2-LA concentrations on day 28 fluctuated slightly (less than 2-fold) during the 12- or 24-hour dosing interval, and then fell slowly, with a mean terminal half-life of 42.3±9.8 hours in cohort 1 and 30.3±18.3 hours in cohort 2. When the daily dose was increased 5-fold from 1.8 g to 9.0 g, the 24-hour areas under the curve (AUCs) on day 28 rose from 1,527±445 to 6,800±918 μg*h/mL (approximately 4-fold), suggesting that D2-LA PK were dose proportional in these subjects.

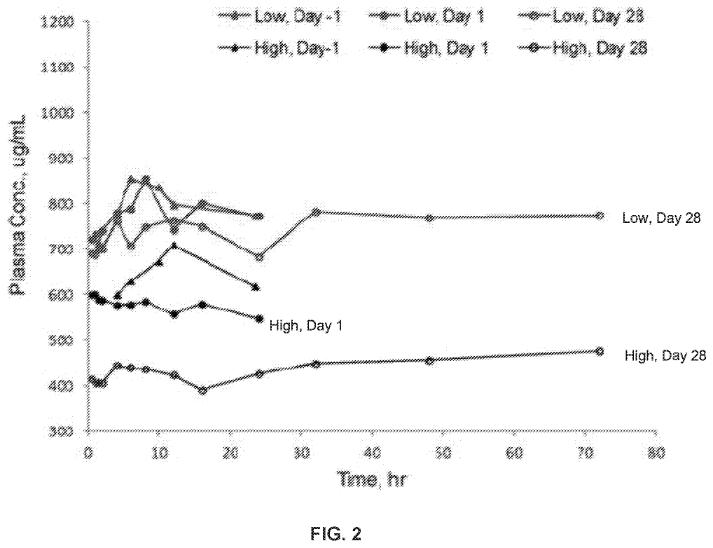

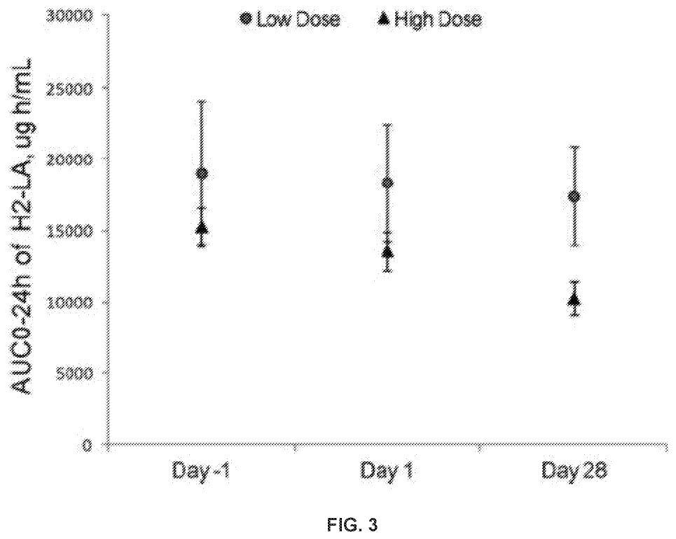

Plasma concentrations of LA on days 1 and 28 did not appear to change from the predose baseline levels after treatment with 11,11-D2-linoleic acid ethyl ester at the low (1.8 g QD) dose. In contrast, after the high (4.5 g BID) dose, the plasma LA (nondeuterated; H2-LA) levels after 28 days of 11,11-D2-linoleic acid ethyl ester treatment appeared to be lower than those at baseline and day 1 in all active treatment subjects (FIG. 2). The day 28 plasma AUC for H2-LA (10,275 μg*h/mL) was approximately 67% of baseline AUC (15,310 μg*h/mL; FIG. 3). This apparent decline in plasma H2-LA concentrations is consistent with the observed increase in the fraction of D2-LA in the total plasma LA pool, which was between 5.0% and 12.1% in cohort 1 (low dose) and between 34.5% and 47.6% in cohort 2 (high dose).

In the low-dose group, plasma concentrations of D2-AA were not detectable before dosing and remained below quantitation on day 1 of the low-dose treatment. By day 28, D2-AA concentrations in plasma had risen to detectable levels in 11,11,D2-linoleic acid ethyl ester treated subjects. Mean concentrations after the last dose fluctuated with a possible circadian pattern, with maxima at 6 hours (3.86 μg/mL) and 32 hours (5.53 μg/mL). In subjects on high dose 11,11,D2-linoleic acid ethyl ester, predose baseline and day 1 concentrations were below or slightly above the LOQ (1 μg/mL) in all subjects, suggesting little or no exposure to D2-AA. By day 28, however, D2-AA concentrations in plasma had risen to robust and potentially therapeutic levels in the high-dose treated subjects. Plasma D2-AA appeared to remain relatively steady with a mean of around 22 μg/mL during the 72-hour period after the last dose, suggesting that high-dose treatment resulted in the presence of robust concentrations of D2-AA, after expected initial delays in enzymatic conversion into AA in the liver, in the plasma by day 28. The mean fraction of D2-AA in the total plasma AA pool on day 28 ranged from 9.1% to 12.6% in the high-dose subjects.

The ability of 11,11-D2-linoleic acid ethyl ester to enter the cellular membrane compartment was assessed by measuring 11,11-D2-linoleic acid ethyl ester and LA concentration in RBCs during the 28-day period. The administration of 11,11-D2-linoleic acid ethyl ester did not appear to affect the levels of LA in RBCs, with similar levels observed in the control and active treatment subjects. RBC levels of D2-LA were not detectable before dosing in all subjects and remained so at 1 day after the first dose in more than half of the active treatment subjects at both dose levels. However, by day 14, all subjects had measurable concentrations of D2-LA in the RBC compartment, and RBC concentrations continued to rise through the day 28. The mean day 28 RBC concentrations of D2-LA were 14.6±8.7 μg/mL for cohort 1 and 26.1±19.2 μg/mL for cohort 2. The RBC concentration of D2-LA, expressed as a percentage of total LA (D2-LA plus LA), rose to a mean value of 6.52% in cohort 1 and 25.8% in cohort 2.

### Functional Assessments

Four protocol-defined outcomes (peak workload, VO₂max, FARS-Neuro, and T25FW) were examined to explore for possible endpoints for future studies in FRDA. Nonparametric statistics were used to compare the results of those receiving 11,11-D2-linoleic acid ethyl ester to comparator. A modified intention-to-treat (mITT) population was used for the VO₂max and peak workload testing analysis because 2 subjects were unable to complete the exercise test. One subject was unable to establish required pedal cadence, and the test was terminated at the conclusion of the warm-up at baseline and day 27 and 1 subject had to stop the day 27 testing because the subject's knees were hitting together and the subject was unable to maintain the RPM. A significant difference between study drug and comparator was observed for peak workload (P=0.008). After Bonferroni adjustment for testing the four outcomes, the change in peak workload between study drug and comparator remained significant (P=0.032). This Bonferroni adjustment is standard in early-stage trials to analyze both prospective or post-hoc data from multiple exploratory efficacy endpoints in order, to determine the most suitable primary endpoint for subsequent pivotal trials.

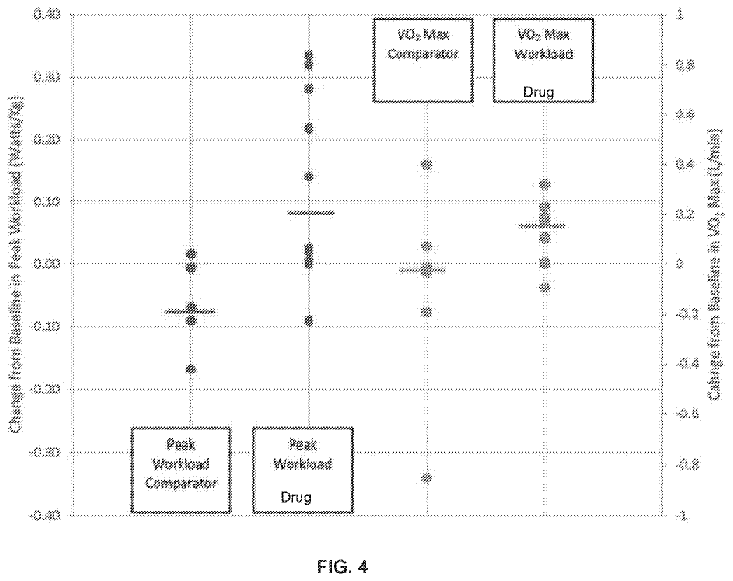

Specifically, CPET testing showed an improvement from baseline to day 28 in peak workload of 0.16 watts/kg in subjects taking study drug (n=10) versus comparator (n=6; P=0.008, Mann-Whitney ranksum test, 25.7% change in the drug group at study end-F4 point; FIG. 4). The mITT analysis excluded 2 subjects in the 11,11,D2-linoleic acid ethyl ester treatment group who were unable to either start or complete the CPET test protocol. However, full ITT analysis including these 2 subjects also resulted in significant improvement in workload (P=0.021). The study also showed an improvement from baseline to day 28 in peak oxygen consumption (VO₂max). The median change from baseline for comparator versus drug was −0.02 versus 0.14 L/min (P=0.116; 17.5% change between drug and comparator, FIG. 4).

Assessment of FARS-Neuro scoring showed an improvement between baseline and day 28 in both groups. There was a median decrease of 2.80 FARS points in the placebo group and a median decrease of 4.75 FARS points in the drug treatment group (a difference of a 1.95 FARS points decrease between both groups; P=0.348 IT). These data included a single subject in the placebo group who experienced a very high improvement in FARS-Neuro (by 11.8 points). Excluding this subject, there was a median decreases of 4.75 FARS-Neuro points in the drug treatment group compared to a median decrease of 1.75 points in the placebo group (P=0.09). Using an electronic motion sensing device mounted on the ankle of each subject, subject stride times, averaged over the 25-foot timed walking protocol, and reported as a stride speed (stride time)⁻¹ improved by 7% (P=0.15 ITT). Conversely, traditionally measured (by stopwatch) T25FW⁻¹ time showed no signal of significance (P=0.88).

Correlations were noted between multiple progression parameters measured cross-sectionally across all patient encounters. Comparisons of peak workload, peak oxygen consumption, FARS Neuro point, and T25FW showed consistent cross-correlation.

### DISCUSSION

In this double-blind, placebo-controlled trial, 11,11-D2-linoleic acid ethyl ester was shown to be safe and tolerable in FRDA patients. The drug was found to be promptly absorbed and well tolerated, achieving steady state in the plasma and in RBCs within 28 days. Increasing the daily D2-LA dose 5-fold resulted in a 4-fold increase in plasma D2-LA exposure, suggesting that D2-LA PK were dose proportional in these patients. 11,11,D2-linoleic acid ethyl ester was converted into an active, stabilized form of D2-AA which also achieved steady state in plasma and within RBCs, consistent with preclinical studies. Functional testing as assessed by peak workload achieved during CPET testing also improved significantly compared to placebo. Other measures of function, including VO₂max, showed improvement trends as well.

Overall, 11,11,D2-linoleic acid ethyl ester was safe with no major adverse effects observed. Although gastrointestinal AEs were observed in both subjects randomized to high-dose 11,11-D2-linoleic acid ethyl ester as well as to comparators. These findings are consistent with large doses of dietary oils rather than study drug alone (e.g., fish oils). This AE can be avoided by reducing the dose of study drug and/or dividing the dose between two or three meals.

The presence of oxidative stress in FRDA has been well documented. The reduction in frataxin in FRDA results in mitochondrial iron accumulation and the formation of reactive oxidative species and LPO of the inner mitochondrial membrane. The high concentration of PUFAs in the inner mitochondrial membrane makes it especially vulnerable to LPO. LA is the primary form of mitochondrial membrane PUFA in all peripheral tissues, along with ARA. Whereas a few grams of LA are needed to compete with dietary inputs of that fat, recommended dietary amounts of ARA are 10-fold lower. Thus even modest conversion of stabilized linoleic to arachidonic acids can be sufficient to treat central nervous system (CNS) tissues in neurodegenerative disease states like FRDA.

The key step in LPO of the mitochondrial membrane is hydrogen abstraction from a bis-allylic site of PUFAs. Deuteration of these bis-allylic sites slows peroxidation and protects against further oxidative damage and formation of toxic by-products. One study evaluated the effect of oxidative stress in LPO in FRDA using several cell models that were treated with deuterated PUFAs at bis-allylic sites. FRDA cells that were oxidatively stressed were protected by deuterated linoleic and alpha-linolenic acids. See Cotticelli et al., Redox Biol 2013, 1:398-404.

Dietary LA and 11,11-D2-linoleic acid ethyl ester are not utilized by the brain, but are first converted by the body into arachidonic and 13,13-D2-arachidonic acids (D2-AA), which the brain takes up. Any activity against neurological damage is believed to rely on the successful conversion of 11,11,D2-linoleic acid ethyl ester into an active stabilized form of AA, that is then taken up into the CNS. The current study demonstrated that 11,11,D2-linoleic acid ethyl ester was absorbed and equilibrated with plasma and cellular levels of both the drug itself, as well as its metabolite (D2-AA) in patients with FRDA. Hence, 11,11-D2-linoleic acid ethyl ester is both a drug, replacing LA in peripheral tissues with a membrane fat stabilized against oxidation, and a prodrug for stabilized D2-AA, which is formed from 11,11,D2-linoleic acid ethyl ester in the liver, and has direct access to the brain as D2-AA.

Progression in FRDA is most commonly measured using the FARS-Neuro Score. Changes in this score are usually measured over longer periods of time, given that FRDA patients typically decline an average of 2 points per year. In the current study, FARS-Neuro scores understandably did not reach significance between drug and comparator over 28 days. However, there was a significant improvement in workload capacity at endpoint relative to baseline for the treatment group. Clear correlations between multiple progression parameters measured cross-sectionally across all patient encounters were observed in this study (comparisons of peak workload, VO₂ peak, FARS Neuro Score, and T25FW showed consistent cross-correlation). These appear to be legitimate measures of disease severity and are correlated to well-studied measures such as FARS-Neuro. Importantly, CPET data are potentially of great value in assessing functional capacity of patients both through measures of maximal capability (i.e., VO₂ peak and peak workload) that submaximal capability (i.e., ability to perform activities of daily living). That is, increased peak metabolic and workload capabilities should result in improved submaximal capacity that is made manifest in completing functional activities with reduced exercise and less fatigue.

What is claimed is:

1. A method of treating a subject having Friedreich's ataxia, comprising:
selecting a subject having Friedreich's ataxia; and
administering an effective amount of 11,11-D2-linoleic acid or an ester thereof to the subject in need thereof.

2. The method of claim 1, wherein the subject having Friedreich's ataxia has mutations in the frataxin (FXN) gene.

3. The method of claim 2, wherein the mutations comprise an expanded GAA repeat mutation in the FXN gene.

4. The method of any one of claims 1 to 3, wherein the subject has a FARS-Neuro score from about 20 to about 100.

5. The method of any one of claims 1 to 3, wherein the subject has a body mass index (BMI) from about 15 kg/m² to about 35 kg/m².

6. The method of anyone of claims 1 to 5, wherein the 11,11-D2-linoleic acid ester is an ethyl ester.

7. The method of anyone of claims 1 to 6, wherein the amount of 11,11-D2-linoleic acid or the ester thereof is from about 1 g to about 10 g.

8. The method of claim 7, wherein the amount of 11,11-D2-linoleic acid or the ester thereof is from about 2 g to about 5 g.

9. The method of any one of claims 1 to 8, wherein 11,11-D2-linoleic acid or the ester thereof is administered once per day.

10. The method of any one of claims 1 to 8, wherein 11,11-D2-linoleic acid or the ester thereof is administered two or more times per day.

11. The method of any one of claims 1 to 10, wherein the amount of 11,11-D2-linoleic acid or the ester thereof is from about 1 g to about 20 g per day.

12. The method of claim 11, wherein the amount of 11,11-D2-linoleic acid or the ester thereof is from about 2 g to about 10 g per day.

13. The method of any one of claims 1 to 12, wherein 11,11-D2-linoleic acid or the ester thereof is administered for at least 2, 3, or 4 weeks.

14. The method of any one of claims 1 to 13, wherein the steady state plasma 24-hour AUC of 11,11-D2-linoleic acid or the ester thereof is from about 1000 μg*h/mL to about 8000 μg*h/mL.

15. The method of any one of claims 1 to 14, further comprising detecting a metabolite of 11,11-D2-linoleic acid or the ester thereof in the plasma.

16. The method of claim 15, wherein the metabolite is 13,13-D2-arachidonic acid.

17. The method of claim 16, wherein the steady state plasma concentration of 13,13-D2-arachidonic acid is from about 2 μg/mL to about 40 μg/mL.

18. The method of any one of claims 1 to 13, further comprising detecting 11,11-D2-linoleic acid or the ester thereof inside the red blood cells (RBCs).

19. The method of claim 18, wherein the steady state RBC concentration of 11,11-D2-linoleic acid or the ester thereof is from about 5 μg/mL to about 75 μg/mL.

20. The method of claim 19, wherein the RBC concentration of 11,11-D2-linoleic acid or the ester thereof is about 5% to about 30% of total linoleic acid or the ester thereof.

21. The method of any one of claims 1 to 20, wherein the method improves one or more parameters selected from the group consisting of VO₂max, FARS-neuro, cardiopulmonary exercise testing (CPEP), peak workload, and timed 25-foot walk (T25FW).

22. The method of any one of claims 1 to 21, wherein the subject also ingests a non-isotopically modified polyunsaturated fatty acid, a non-isotopically modified polyunsaturated fatty acid ester, or a combination thereof.

23. The method of claim 22, wherein the amount of 11,11-D2-linoleic acid or the ester thereof is about 5% or greater of the total amount of the polyunsaturated fatty acids and polyunsaturated fatty acid esters delivered to the subject.

24. The method of claim 22, wherein the amount of the 11,11-D2-linoleic acid or the ester thereof is equal to less than about 1% of the total amount of the polyunsaturated fatty acids and polyunsaturated fatty acid esters delivered to the subject.

25. The method of any one of claims 1 to 24, wherein 11,11-D2-linoleic acid or the ester thereof is administered with food.

26. The method of any one of claims 1 to 24, wherein 11,11-D2-linoleic acid or the ester thereof is administered between meals.

27. The method of any one of claims 1 to 26, wherein 11,11-D2-linoleic acid or the ester thereof is administered with at least one antioxidant.

28. The method of claim 27, wherein the antioxidant is selected from the group consisting of Coenzyme Q, idebenone, mitoquinone, mitoquinol, vitamin E, and vitamin C, and combinations thereof.
